(19) United States
(12) Patent Application Publication
(10) Pub. No.: US 2011/0014294 A1
(43) Pub. Date: Jan. 20, 2011

(54) STIMULATION OF OCULAR RETROBULBAR BLOOD FLOW USING OCULAR IRRITANTS

(75) Inventors: Yossi Gross, Moshav Mazor (IL); Steve B. Koevary, Newton, MA (US)

Correspondence Address: FENNEMORE CRAIG, 3003 NORTH CENTRAL AVENUE, SUITE 2600, PHOENIX, AZ, 85012, US

(73) Assignee: PHARMALIGHT INC., Wilmington, DE (US)

(21) Appl. No.: 12/920,458

(22) PCT Filed: Mar. 2, 2009
(86) PCT No.: PCT/IB2009/050834

§ 371 Date: Oct. 6, 2010

Related U.S. Application Data
(60) Provisional application No. 61/033,076, filed on Mar. 3, 2008.

(51) Int. Cl. A61K 9/12; A61K 31/7048; A61K 31/14; A61P 27/06; A61P 27/02; A61P 9/12; A61P 9/10; A61P 29/00; A61P 3/02

(52) U.S. Cl. 424/489; 514/26; 514/643

(57) ABSTRACT

Disclosed are uses of an ocular irritant such as saponin in stimulating the retrobulbar blood flow of an eye. Disclosed are also methods of treatment including administration of a pharmaceutical composition including an ocular irritant to an eye, for example as a mist, in order to stimulate the retrobulbar blood flow. In some embodiments, the stimulation of the retrobulbar blood flow has a beneficial effect.

### RELATED APPLICATIONS

The present application gains priority from U.S. Provisional Patent Application No. 61/033,076 filed 3 Mar. 2008 which is included by reference as if fully set forth herein.

Some embodiments of the present invention are related to the teachings of PCT Patent Application No. PCT/IL2006/000145 filed 6 Feb. 2006 and published as WO2006/082588, which claimed the benefit of U.S. Provisional Patent Application No. 60/650,144 filed on 7 Feb. 2005. The contents of the above Applications are incorporated by reference as if fully set forth herein.

### FIELD AND BACKGROUND OF THE INVENTION

The present invention relates to the field of medicine and more particularly in some embodiments to methods, uses, compositions and devices relating to the stimulation of ocular retrobulbar blood flow. In some embodiments, the invention relates to the ocular administration of a pharmaceutical composition including an ocular irritant as a mist which, in some embodiments, is effective in stimulating retrobulbar blood flow.

The bulb of the eye (eyeball) is contained in the cavity of the orbit. Associated with the eye are certain accessory structures such as the muscles, fasciæ, eyelids, conjunctiva, and lacrimal apparatus. Only the surface of the anterior part of the eye, including the corneal epithelium and part of the episcleral conjunctiva, are exposed to the environment. The mucosa of the conjunctiva provide a protective interface between the eye and accessory structures. The exposed anterior surface of the eye is continuously washed by tear fluid. The nasolacrimal duct drains tears and other substances from the eye to be absorbed by a layer of mucosal membrane.

The eye is provided with blood through various retrobulbar arteries. The eye is extremely sensitive to any disruptions of its blood supply, which occur more frequently with age. Most disruptions of blood supply result at least partly from occlusion, for example due to atherosclerosis or an embolus, but may also occur as a result of inflammation of the blood vessels (vasculitis, such as temporal arteritis), inflammation of the optic nerve, infection in or around the eye, clotting disorders, damage from radiation, and injury to the eye. Disruption of blood flow to the eye generally results in vision loss, usually in one eye, which may be total or partial.

Reduced blood flow to the eye through the retrobulbar arteries has been associated with a number of ocular conditions, for example insufficient retrobulbar blood flow, diabetic retinopathy; open angle glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

Diabetic retinopathy is a complication of diabetes which results from damage to the blood vessels of the retina due to hyperglycemia-induced pericyte death and thickening of the basement membrane, leading to incompetence of the vascular walls, which may lead to blindness.

Open angle glaucoma is a disease distinguished by an increase in pressure inside the eye caused by gradual blockage of aqueous outflow due to clogging of the drainage system or over-production of aqueous fluid, and resulting in damage to the optic nerve and to the retina.

Ocular hypertension refers to any condition in which intraocular pressure is higher than normal, which may be due to, for example, traumatic hyphema, orbital edema, postoperative viscoelastic retention, intraocular inflammation, corticosteroid use, pupillary block, or idiopathic causes.

Macular degeneration is a medical condition usually of older adults which results in a loss of vision in the center of the visual field (the macula) because of damage to the retina. It occurs in “dry” and “wet” forms. The “dry” form results from atrophy to the retinal pigment epithelial layer below the retina, which causes vision loss through loss of photoreceptors (rods and cones) in the central part of the eye. The “wet” form causes vision loss due to abnormal blood vessel growth in the choriocapillaries, through Bruch's membrane, ultimately leading to blood and protein leakage below the macula. Bleeding, leaking, and scarring from these blood vessels eventually cause irreversible damage to the photoreceptors and rapid vision loss if left untreated.

Ocular ischemic syndrome is caused by internal carotid artery atheromatous ulceration and stenosis at the bifurcation of the common carotid artery.

Giant cell arteritis is an inflammatory disease of blood vessels, often in the head. When the inflammation affects the blood supply to the eyes, blurred vision or sudden blindness may occur.

Eye occlusions, also called eye strokes, are when blood flow to important eye structures is blocked, for example, by a clot. For example, central retinal artery occlusion (CRAO) and central retinal vein occlusion (CRVA) are when the artery or vein associated with the retina become occluded, potentially leading to complete loss of vision.

Ischemic optic neuropathy (both anterior and posterior ischemic optic neuropathy) is the loss of vision resulting by damage to a portion of the optic nerve due to obstruction of blood flow to the nerve (i.e., ischemia). In optic neuritis, inflammation of the optic nerve, especially of the myelin covering of the optic nerve, damages the nerve and may adversely affect vision. Optic neuritis is related to, associated with or may be caused by auto-immune diseases, multiple sclerosis, neuromyelitis optica, neuroretinitis, bacterial infections (e.g., Lyme's disease, cat scratch fever, syphillis), viral infections (e.g., HIV, hepatitis B, herpes), cranial arteritis, diabetes, drugs (e.g., ethambutol), radiation therapy, tumors, nutritional deficiencies, toxins and others.

It would be useful to be able to stimulate retrobulbar blood flow, for example in order to treat conditions associated with insufficient retrobulbar blood flow.

### SUMMARY OF THE INVENTION

Some embodiments of the present invention are related to the unexpected discovery that ocular irritants, when administered as a mist, stimulate retrobulbar blood flow to a clinically-useful extent, for example, in some embodiments sufficient to be useful for treating a condition.

Thus, according to an aspect of some embodiments of the invention, there is provided a method of treatment, comprising administering to an eye of a subject suffering from a condition an effective amount of a pharmaceutical composition including an ocular irritant in an ophthalmically-acceptable carrier as a mist whereby the ocular irritant stimulates retrobulbar blood flow of the eye, thereby treating the condition.

According to an aspect of some embodiments of the invention, there is also provided the use of a pharmaceutical composition comprising an effective amount of an ocular irritant in an ophthalmically-acceptable carrier as a mist administered to an eye of a subject for the treatment of a condition susceptible to stimulation of retrobulbar blood flow.

Generally, the pharmaceutical composition is administered as a mist to the anterior part of an eye of the subject.

According to an aspect of some embodiments of the invention, there is also provided the use of an ocular irritant together with an ophthalmically-acceptable carrier in the preparation of a pharmaceutical composition for administration as a mist to the eye for the treatment of a condition susceptible to stimulation of retrobulbar blood flow. In some embodiments, the condition is selected from the group consisting of insufficient retrobulbar blood flow, diabetic retinopathy, open angle glaucoma, ocular hypertension, macular degeneration, ocular ischemic syndrome, giant cell arteritis, eye occlusions, central retinal artery occlusion (CRAG), central retinal vein occlusion (CRVA), ischemic optic neuropathy, optic neuritis, neuromyelitis optica and neuroretinitis.

According to an aspect of some embodiments of the invention, there is also provided a method of treatment, comprising: a) providing a pharmaceutical composition consisting essentially of an ocular irritant and an ophthalmically-acceptable carrier; b) generating a mist of the pharmaceutical composition; and c) contacting the mist with a posterior surface of an eye of a subject in need thereof. In some embodiments, contacting of the mist with the posterior surface of the eye leads to depositing of an amount of the ocular irritant on the posterior surface effective in stimulating the retrobulbar blood flow of the eye.

According to an aspect of some embodiments of the invention, there is also provided the use of a mist for ophthalmic delivery of a pharmaceutical composition consisting essentially of an ocular irritant and an ophthalmically-acceptable carrier to a subject in need thereof.

In some embodiments, the need is for treating a condition. By treating a condition is meant, for example, curing a condition, preventing a condition, treating symptoms of a condition, curing symptoms of a condition, ameliorating symptoms of a condition, treating effects of a condition, ameliorating effects of a condition, and preventing results of a condition. In some embodiments, the need is at least partially satisfied by stimulation of the retrobulbar blood flow of the eye by the ocular irritant.

In some embodiments, the condition is selected from the group consisting of insufficient retrobulbar blood flow, diabetic retinopathy, open angle glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

In some embodiments of the methods or uses, the subject is a human. In some embodiments of the methods or uses, the subject is a non-human animal.

According to an aspect of some embodiments of the invention, there is also provided a pharmaceutical composition, consisting essentially of an ocular irritant and an ophthalmically-acceptable carrier, the composition configured for stimulating retrobulbar blood flow in an eye to which delivered, and further configured for ocular administration as a mist.

According to an aspect of some embodiments of the invention, there is also provided a device, comprising: a) a composition-reservoir configured to be functionally associated with a nebulizer; and b) a pharmaceutical composition consisting essentially of an ocular irritant and an ophthalmically-acceptable carrier contained within the reservoir, the pharmaceutical composition configured for stimulating retrobulbar blood flow in an eye to which administered as a mist. In some embodiments, the device further comprises: c) a nebulizer suitable for ophthalmic administration of a composition, configured to nebulize composition contained in a functionally associated composition-reservoir to generate an ophthalmically-administrable mist.

In some embodiments of the uses, methods, devices or pharmaceutical compositions, the pharmaceutical composition consists essentially of the ocular irritant.

In some embodiments of the uses, methods, devices or pharmaceutical compositions, the pharmaceutical composition is substantially devoid of an active pharmaceutical ingredient.

In some embodiments of the uses, methods, devices or pharmaceutical compositions, the pharmaceutical composition comprises at least one ocular irritant.

In some embodiments of the uses, methods, devices or pharmaceutical compositions, the pharmaceutical composition comprises at least two different ocular irritant.

In some embodiments of the uses, methods, devices or pharmaceutical compositions, the pharmaceutical composition comprises a single ocular irritant.

In some embodiments of the uses, methods, devices or pharmaceutical compositions, the pharmaceutical composition comprises saponin.

In some embodiments of the uses, methods, devices or pharmaceutical compositions, the pharmaceutical composition comprises benzalkonium chloride.

In some embodiments of the uses, methods, devices or pharmaceutical compositions, the pharmaceutical composition comprises at least about 0.001% by weight of the ocular irritant.

In some embodiments of the uses, methods, devices or pharmaceutical compositions, the pharmaceutical composition comprises at least about 0.01% by weight of the ocular irritant.

In some embodiments of the uses, methods, devices or pharmaceutical compositions, the pharmaceutical composition comprises at least about 0.1% by weight of the ocular irritant.

Unless otherwise defined, all technical and scientific terms used herein have the same meaning as commonly understood by one of ordinary skill in the art to which the invention pertains. In case of conflict, the patent specification, including definitions, will control.

As used herein, the indefinite articles “a” and “an” mean “at least one” or “one or more” unless the context clearly dictates otherwise.

### DESCRIPTION OF SOME EMBODIMENTS OF THE INVENTION

Aspects of the present invention relate to the administration of a pharmaceutical composition comprising an ocular irritant to the eye as a mist in order to stimulate retrobulbar blood flow. Some embodiments of the present invention relate to methods, uses, devices and compositions relating to the administration of a pharmaceutical composition comprising an ocular irritant and an ophthalmically-acceptable carrier as a mist to an eye of a subject.

In PCT patent publication WO2006/082588 of the Inventor is disclosed that when a pharmaceutical composition including an ocular irritant such as a penetration enhancer and an active pharmaceutical ingredient such as a protein or peptide is administered to the surface of the anterior part of the eye as a mist, the intensity of irritation caused by the penetration enhancer is reduced.

It has now surprisingly been found, that ocular irritants such as penetration enhancers, when administered to the eye as a mist, stimulate retrobulbar blood flow to a clinically useful extent, that is to say, in some instances to an extent sufficient that is useful for treating a condition susceptible to stimulation of retrobulbar blood flow. This finding is especially surprising in view of the fact that as shown in the Examples section below, ocular irritants, when administered as eye drops, have no effect on retrobulbar blood flow, or even reduce retrobulbar blood flow slightly.

Without wishing to be bound to a single hypothesis, it is considered that the effect of ocular irritants in increasing retrobulbar blood flow is mediated by the sphenopalatine (pterygopalatine) ganglion, which is a parasympathetic ganglion that sends post-ganglionic, parasympathetic fibers to the lachrymal gland. Ocular irritation triggers the tear reflex. The fibers that form the efferent limb of this reflex, in addition to stimulating tear production, also induce an increase in retrobulbar blood flow. Furthermore, stimulation of endothelial muscarinic receptors, which are known to induce the release of the vasodilator nitric oxide, may be involved in the upregulation of retinal blood flow by acetylcholine released from postganglionic cholinergic neurons. It has further been shown that stimulation of the sensory nerves of the rabbit eye caused an increase in blood flow through the iris by a mechanism that seemed to involve substance P and calcitonin gene related peptide.

Thus, according to an aspect of some embodiments of the invention, there is provided a method of treatment, comprising administering to an eye of a subject suffering from a condition an effective amount of a pharmaceutical composition including an ocular irritant in an ophthalmically-acceptable carrier as a mist so as to stimulate retrobulbar blood flow of the eye, thereby treating the condition. It is believed that the ocular irritant in the pharmaceutical composition stimulates retrobulbar blood flow of the eye, thereby treating the condition.

Thus, according to an aspect of some embodiments of the invention, there is also provided the use of a pharmaceutical composition comprising an effective amount of an ocular irritant in an ophthalmically-acceptable carrier as a mist administered to an eye of a subject for the treatment of a condition susceptible to stimulation of retrobulbar blood flow.

In some embodiments of the invention, administration of a pharmaceutical composition including an ocular irritant as a mist stimulates retrobulbar blood flow but also occurs with reduced, minimal or no irritation to the eye.

Herein, the term “mist” refers to a cloud of particles having a mean particle diameter of less than about 20 microns, less than about 10 microns, less than about 8 microns, less than about 5 microns, less than about 3 micron and even less than about 1 micron. Mists are formed, for example, with a nebulizer.

Herein, the term “nebulizer” is understood to mean a device or a part of a device that converts a substance, e.g., a solid, gel, liquid, solution, suspension, ointment, pharmaceutical composition, into a mist.

Some embodiments of the present invention may be implemented using any nebulizing device known in the art for ophthalmic administration of a pharmaceutical composition, especially embodiments of devices of described in the PCT patent publication WO2006/082588 of the Inventor, the nebulizer device described in U.S. Pat. No. 6,748,944 or an ophthalmic delivery device by Optimyst, Llc (West Islip, N.Y., USA) as described in Am J Opthalmol 2007 114, 137-139 and in WO 2008/094481.

The teachings of the invention are generally implemented in the context of treating a need of a human or non-human animal subject. In some embodiments, the need is treating a condition. By treating a condition is meant, for example, curing a condition, preventing a condition, treating symptoms of a condition, curing symptoms of a condition, ameliorating symptoms of a condition, treating effects of a condition, ameliorating effects of a condition, and preventing results of a condition.

Generally, conditions treated by embodiments of the invention are conditions susceptible to stimulation of the retrobulbar blood flow of an eye of a subject, for example, insufficient retrobulbar blood flow, diabetic retinopathy, open angle glaucoma, ocular hypertension, macular degeneration, and ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

Herein, the term “ocular irritant” refers to a material that leads to irritation of the eye upon ordinary contact therewith, e.g., when a solution including the material is administered as drops to an eye. In some embodiments of the invention, a pharmaceutical composition comprises at least one ocular irritant. In some embodiments of the invention, the pharmaceutical composition comprises at least two different ocular irritants. In some embodiments of the invention, the pharmaceutical composition comprises a single ocular irritant.

Some ocular irritants are known penetration enhancers, materials that increase the amount or rate of absorption into the body of a substance coadministered therewith. In some embodiments, an ocular irritant used in implementing the invention is a penetration enhancer. Penetration enhancers are materials that transiently increase the permeability of the corneal epithelium or conjunctiva to facilitate API (active pharmaceutical ingredient) penetration therethrough. The use of known percutaneous penetration enhancers in ophthalmic compositions has been proposed (see Sasaki et al. Crit. Rev. Ther. Drug Carrier Syst. 1999, 16, 85-146) but is not generally used due to observations of irritation and corneal and conjunctival injury caused by known penetration enhancers, see Saettone et al. Int. J. Pharm. 1996, 142, 103-113 and Furrer et al. AAPS Pharm. Sci. 2002, 4(1), 1-5).

Ocular irritants can be classified as being inherently highly irritating to the eye or as being mildly irritating to the eye.

An inherently highly-irritating ocular irritant that is a known penetration enhancer and that is useful as an ocular irritant component of a pharmaceutical composition for implementing some embodiments of the invention is saponin (including saponin derivatives).

An inherently highly-irritating ocular irritant that is a known penetration enhancer and that is useful as an ocular irritant component of a pharmaceutical composition for implementing some embodiments of the invention is benzalkonium chloride.

An inherently highly-irritating ocular irritant that is a known penetration enhancer and that is useful as an ocular irritant component of a pharmaceutical composition for implementing some embodiments of the invention is sodium caprate.

Other inherently highly irritating ocular irritants that are known penetration enhancers and that are useful for implementing the teachings of the invention as components of an embodiment of a composition of the invention include, but are not limited to BL-9, deoxycholic acid, digitonin, escin, fusidic acid, fusidate, fusidic acid derivatives, sodium deoxycholate, acetone, acyl lactylates, acyl peptides, acylsarcosinates, alcohols, alkanolamine salts of fatty acids, alkyl benzene sulphonates, alkyl ether sulphates, alkyl sulphates, allantoin, anionic surface-active agents, 1-substituted azacycloheptan-2-ones, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl)ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, glycerol monolaurate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, lecithin, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sorbitan monooleate, sorbitan monolaurate, sugar esters, sulphosuccinate, tetrahydrofuran, tetrahydrofurfural alcohol, transcutol, triethanolamine dodecyl benzene sulphonate, triethanolamine oleate, urazole, urea and derivatives, esters, salts and mixtures thereof.

Ocular irritants that are penetration enhancers that are considered mildly irritating at low concentrations, but highly irritating at high concentrations that are useful for implementing some embodiments of the invention as components of an embodiment of a composition of the invention include, but are not limited to ammonium glycyrrhizide, Brij 35, Brij 78, Brij-98, cetylpyridium chloride, chenodeoxycholic acid, cholate, cholic acid, decamethonium, decamethonium bromide, dimethyl sulphoxide, EDTA and disodium EDTA, glycocholate, glycocholic acid, glycodeoxycholic acid, glycyrrhizic acid, paraben, polyoxyethylene, polyoxyethylene ethers of fatty acids such as polyoxyethylene 4-, 9-, 10-, and 23-lauryl ether, polyoxyethylene 10- and 20-cetyl ether, polyoxyethylene 10- and 20-stearyl ether, polyoxyethylated castor oil, polyoxyethylene monolaurate, polyoxyethylene sorbitans such as polyoxyethylene sorbitan monolaurate, polyoxy:polyoxyethylene stearate, polyoxypropylene 15 stearyl ether, sodium cholate, sodium glycocholate, sodium taurocholate, sodium glycodeoxycholate, sodium taurodeoxycholate, sodium ursodeoxycholate, taurocholic acid, taurodeoxycholic acid, TWEEN 20, urosdeoxycholic acid, and derivatives, esters, salts and mixtures thereof in a greater than accepted concentration.

Some embodiments of a pharmaceutical composition suitable for implementing the teachings of the invention include an ocular irritant in an ophthalmically-acceptable carrier and optionally other ingredients.

A pharmaceutical composition suitable for implementing the teachings of the invention may include any suitable concentration of ocular irritant, that is to say a concentration that is sufficient to provide a desired degree of retrobulbar blood flow stimulation when appropriately administered. That said, in some embodiments, the ocular irritant makes up at least about 0.001%, at least about 0.01%, at least about 0.05%, at least about 0.1%, at least about 0.2%, at least about 0.5%, at least about 1% and even at least about 2% by weight of the pharmaceutical composition.

The concentration of a specific ocular irritant in any specific embodiment of a pharmaceutical composition is dependent on a number of factors including solubility of the ocular irritant in the carrier, the need to produce a mist from the composition and clinical considerations. Determination of the concentration of a specific ocular irritant needed in a specific embodiment of a pharmaceutical composition is within the capability of one skilled in the art in light of the disclosure provided herein.

Ophthalmically-acceptable carriers are generally sterile, essentially free of foreign particles, and generally have a pH in the range of 5-8. Preferably, the pH is as close to the pH of tear fluid (7.4) as possible. In some embodiments, an opthalmically-acceptable carrier of a pharmaceutical composition is isotonic. Ophthalmically-acceptable carriers are, for example, sterile isotonic solutions such as isotonic sodium chloride or boric acid solutions. Such carriers are typically aqueous solutions contain sodium chloride or boric acid. Also useful are phosphate buffered saline (PBS) solutions. Additional useful carriers as well as specific examples of suitable carriers are described in the Examples, below.

In some embodiments, the pharmaceutical composition consists essentially of an ocular irritant, that is to say, substantially all the retrobulbar blood flow-stimulating effect of the composition is caused by the ocular irritant.

In some embodiments of the invention, the composition is substantially devoid of an active pharmaceutical ingredient, the beneficial effect of the composition being produced substantially entirely by the stimulation of retrobulbar blood flow by the ocular irritant.

In some embodiments, a composition of the present invention includes, in addition to the ocular irritant in a ophthalmically-acceptable carrier, at least one additional component. It is important to note that in some cases a specific additional component also serves as a component of the carrier or serves two or more additional functions. Typical additional components include but are not limited to bioadhesives, buffering agents, chelating agents, humectants, pH-adjusting agents, preservatives, solubilizers, viscosity modifiers and vitamins.

In some embodiments of the present invention, a composition includes a pH-adjusting agent. Suitable pH-adjusting agents include but are not limited to adipic acid, bodes acid, citric acid, glycine, calcium hydroxide, magnesium aluminometasilicates, hydrochloric acid, lactic acid, phosphoric acid, sodium hydroxide, sorbic acid, sulfuric acid and tartaric acid, derivatives thereof, salts thereof or combinations thereof.

In some embodiments of the present invention, a composition includes a buffering agent. Suitable buffering agents include but are not limited to borate buffers, citrate buffers, acetic acid/sodium acetate buffers and a phosphoric acid/sodium phosphate buffers.

In some embodiments of the present invention, a composition includes a viscosity modifier. A suitable viscosity modifier is ethanol.

In some embodiments of the present invention, a composition includes a bioadhesive, especially a bioadhesive polymer. Suitable bioadhesives include but are not limited to polyvinyl alcohol, thiolated poly acrylic acid, carbomer and gellan gum.

In some embodiments of the present invention, a composition includes a humectant. Suitable humectants include but are not limited to ammonium lactate, guanidine, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine and acetamide monoethanolamine, urea, or a combination thereof.

In some embodiments of the present invention, a composition include a preservative. Suitable preservatives include but are not limited to alkanols, C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorobutanol, chlorocresol, citric acid, cocoa butter, coconut oil, diazolidinyl urea, diisopropyl adipate, dimethyl polysiloxane, DMDM hydantoin, disodium EDTA (ethylenediamine tetraacetate), EDTA salts, EDTA fatty acid conjugates, ethanol, fatty acids, fatty alcohols, hexadecyl alcohol, hydroxybenzoate esters, iodopropynyl butylcarbamate, isononyl iso-nonanoate, isothiazolinone, jojoba oil, lanolin oil, methylparaben, mineral oil, oleic acid, olive oil, parabens, polyethers, polyoxypropylene butyl ether, polyoxypropylene cetyl ether, potassium sorbate, propylene glycols, propylparaben, silicone oils, sodium perborate, sodium propionate, sodium benzoate, sodium bisulfite, sorbic acid, sorbates, stearic fatty acid, vitamin E, vitamin E acetate and derivatives, esters, salts and mixtures thereof.

In some embodiments of the present invention, a composition includes a solubilizer. Suitable solubilizers include but are not limited to citric acid, ethylenediamine-tetraacetate, sodium meta-phosphate, succinic acid, urea, cyclodextrin, polyvinylpyrrolidone, diethylammonium-ortho-benzoate, micelle-forming solubilizers, TWEENS, SPANS, polyoxyethylene sorbitan fatty acid ester, polyoxyethylene n-alkyl ethers, n-alkyl amine n-oxides, poloxamers, phospholipids and cyclodextrins.

In some embodiments of the present invention, a composition includes a vitamin. Suitable vitamins include but are not limited to retinoids, vitamin A, retinol, retinal, retinyl palmitate, retinoic acid, tretinoin, iso-tretinoin, vitamin E, tocopherol, vitamin C, L-ascorbic acid, vitamin B₃, niacinamide, alpha hydroxy acids, glycolic acid, lactic acid, tartaric acid, malic acid, citric acid, beta hydroxy acids, salicylic acid, esters thereof and derivatives thereof.

According to an aspect of some embodiments of the invention, there is also provided the use of an effective amount of an ocular irritant together with an ophthalmically-acceptable carrier in the preparation of a pharmaceutical composition for administration as a mist for the treatment of a condition susceptible to stimulation of retrobulbar blood flow.

A pharmaceutical composition is generally prepared by mixing the components together to yield a safe and composition that can be administered as a mist. Formulation of a pharmaceutical composition is within the ability of a person having ordinary skill in the art using techniques with which one of average skill is familiar which are discussed in numerous reference works such as Remington's Pharmaceutical Science 15th Edition.

According to an aspect of some embodiments of the invention, a pharmaceutical composition as described above is provided as a component of a device, contained within a composition-reservoir, where the composition-reservoir is configured to be functionally associated with a nebulizer suitable for generating a mist for ophthalmic administration to an eye. For use, the composition-reservoir is functionally associated with a nebulizer so that when the nebulizer is activated, composition is drawn from the reservoir and nebulized to generate a mist. Once generated, the mist may then be administered to an eye to implement some embodiments of the methods and uses described herein.

In some embodiments, the composition-reservoir of a device is substantially a cartridge configured for reversible association with an appropriate nebulizer, as known in the art.

In some embodiments, a device further comprises a nebulizer suitable for ophthalmic administration of a composition, the nebulizer configured to nebulize composition contained in a composition-reservoir that is functionally associated with the nebulizer to generate an ophthalmically-administrable mist. Suitable nebulizers include nebulizers mentioned herein as well as other suitable nebulizers.

In some embodiments, the reservoir is packaged in a packaging material or is labeled and identified in print, in or on the packaging material, as an ophthalmically deliverable composition as a mist for use for a need, as described above.

Generally, medical personnel such as a doctor prescribing a pharmaceutical composition for use in accordance with the teachings of the invention prescribe a dosage regime including one or more administrations of a dose of the composition over a period of time (e.g., once a day, twice a day, three times a day). The dosage regime is generally chosen to be effective, that is to say sufficient to achieve a desired beneficial effect, e.g., to treat a condition.

Determination of an effective dosage regime is within the capability of a person having ordinary skill in the art in light of the disclosure provided herein for example using techniques with which one of average skill is familiar which are discussed in numerous reference works such as Remington's Pharmaceutical Science 15th Edition.

Factors in determining the dosage regime vary with the type of the condition as well as such factors as the concentration of the ocular irritant, the subject being treated, the severity of the condition, the age, body weight and response of an individual patient and the judgment of the prescribing physician.

### EXAMPLES

Reference is now made to the following examples, which together with the above description, illustrate the invention in a non-limiting fashion.

### Example 1

### Effect of Composition Including Saponin as an Ocular Irritant on Retrobulbar Blood Flow

A laser Doppler retinal blood flow instrument (CLEF 100, Canon Inc., Tokyo, Japan) was used to measure the retinal blood flow rate in the major temporal vein in the right eye at pre-administration baseline of seven albino New Zealand white rabbits (approximately 2 kg), as well as post-administration of a saponin composition described below as drops (three of the seven rabbits) and post-administration of a saponin composition as a mist (two of the seven rabbits), based on the principle of bidirectional laser Doppler velocimetry as described by Costa V P et al in Frog in Retinal and Eye Res 2003, 22, 769-805 or Yoshida A et al in Am. J. Opthalmol. 2003, 135, 356-361, both which are included by reference as if fully set-forth herein. In this instrument, the measuring laser beam is locked onto the target blood vessel during eye movements through an eye-tracking feedback and control system. Doppler-shifted laser light scattered from a retinal vessel is analyzed to determine centerline blood velocity. The blood column diameter is simultaneously measured, and the blood flow rate at the measurement site is automatically calculated, as described by Yoshida A et al in Am. J. Opthalmol. 2003, 135, 356-361.

Venous blood flow, which is known to be directly correlated to arterial blood flow was measured, since retinal veins have a larger diameter than retinal arteries, facilitating better locking of the measuring laser beams onto the target vessel. This is especially the case in rabbits, which retinal arteries are very narrow.

The beam from a red 675-nm diode laser was used for velocity measurement, emitted from a fundus camera-like measuring head. The Doppler-shifted light scattered from the flowing blood cells in the target vessel was detected simultaneously in two directions, separated by a fixed angle. The signals from two photomultiplier tube detectors underwent computer-controlled spectrum analysis, and sequential measurements of velocity were performed automatically. Results were acquired at 50 measurements per second for 2 seconds. A tracking stripe provided by a green 543-nm HeNe laser oriented perpendicular to the target vessel was used to measure the diameter of the retinal vessel. Diameter was determined automatically by computer analysis of the signal produced by the image of the vessel on the CCD sensor using the half height of the transmittance profile to define the blood column edge. Diameter measurements were corrected for the axial length of the eye (operator input) and refractive error of the yee, which is measured by the CLBF itself

Specifically, acoustic coupling gel was placed on the eyelid of each studied eye prior to administration of a composition, the probe of the device positioned and the blood flow velocity in selected veins of the untreated eye was measured to obtain a baseline reading.

After obtaining the baseline reading, a composition including an ocular irritant (1% saponin (CAS 8047-15-2) in PBS) was administered to the anterior surface of the eye. For three rabbits, 10 microliter of the composition were administered using a standard eyedropper. For two rabbits, 350 microliters of the composition were administered as a mist over two minutes using a nebulizer device such as described in PCT publication WO2006/082588 of the Inventor.

Following administration of the composition at time intervals detailed in Table 1 below, acoustic coupling gel was placed on the eyelid of the treated eye, the probe positioned and the blood flow velocity in the same veins was measured.

For two of the 7 rabbits (#10 and #4) to which were administered 50 microliter and 30 microliter of the composition respectively with an eyedropper, only baseline measurements were obtained because the flow in the blood vessels was found to be too blurry to measure.

The retrobulbar blood flow of the remaining five rabbits was measured both pre administration (baseline) and post administration. The baseline measurement of the retrobulbar blood flow of Rabbit #8 was lower than that expected, so the baseline was also measured in the contralateral eye, which showed similar low values.

The results of the measurements are presented in Table 1.

TABLE 1

| | | Vessel | | |
|---|---|---|---|---|
| Rabbit | | diameter | Velocity | Flow |
| # | Treatment | (μm) | (mm/sec) | (μl/min) |
| 10 | Baseline 1 | 135.8 | 20.1 | 8.7 |
| | Baseline 2 | 130.8 | 28.2 | 11.4 |
| | Baseline 3 | 126.2 | 26.4 | 9.9 |
| | Average | 130.9 | 24.9 | 10.0 |
| 4 | Baseline 1 | 128.7 | 12.8 | 5.0 |
| | Baseline 2 | 123.1 | 11.8 | 4.2 |
| | Average | 125.9 | 12.3 | 4.6 |
| 2 | Baseline 1 | 137.5 | 9.2 | 4.1 |
| | Baseline 2 | 133.4 | 12.2 | 5.1 |
| | Average | 135.5 | 10.7 | 4.6 |
| | 20 mins post 10 μl drop | 131.6 | 12.1 | 4.9 |
| | 1% saponin | | | |
| 3 | Baseleine 1 | 130.9 | 20.2 | 8.2 |
| | Baseline 2 | 128.9 | 18.8 | 7.4 |
| | Average | 129.9 | 19.5 | 7.8 |
| | 2 min post 10 μl drop 1% | 130.3 | 13.9 | 5.6 |
| | saponin | | | |
| | 10 min post 10 μl drop 1% | 116.3 | 14.3 | 4.5 |
| | saponin | | | |
| 5 | Baseline | 131.6 | 128.2 | 11.5 |
| | 2 min post 10 μl drop 1% | 128.2 | 13.0 | 5.4 |
| | saponin | | | |
| 7 | Baseline | 122.8 | 22.5 | 8.0 |
| | 17 min post 2 min mist | 134.3 | 31.5 | 13.4 |
| | with 1% saponin (total = | | | |
| | 350 μl) | | | |
| | 25 min post 2 min mist | 127.7 | 31.6 | 12.1 |
| | with 1% saponin (total = | | | |
| | 350 μl) | | | |
| 8 | Baseline 1 (left eye) | 108.1 | 8.1 | 2.2 |
| | 26 min post 2 min mist | 134.4 | 19.4 | 8.3 |
| | with 1% saponin | | | |
| | Baseline 2 (right eye) | 108.5 | 10.7 | 3.0 |

Baseline venous diameters ranged from 108 μm to 138 μm in the seven rabbits. Baseline venous blood speeds ranged from 8.1 mm/s to 28.2 mm/s in the seven rabbits. Baseline blood flow rates ranged from 2.2 μl/min to 11.5 μl/min in the seven rabbits. For each rabbit, the variation of each of these parameters was determined from repeated measurements. The average [Max−Min]/Mean for venous diameter was 4.4%. The average [Max−Min]/Mean for venous blood speed was 18.0%. The average [Max−Min]/Mean for blood flow rate was 19.1%.

In the three rabbits where the composition was administered as drops (#2, #3 and #5), the retrobulbar blood flow decreased on average by 27.5%.

In the two rabbits where the composition was administered as a mist (#7 and #8), the retrobulbar blood flow increased from 8.0 μl/min to 12.8 μl/min in the first rabbit, and from 2.2 μl/min to 8.3 μl/min in the second rabbit.

Though sedated, rabbits to which composition was administered as drops showed obvious signs of ocular irritation that included lid closure and squealing, the latter of which lasted for about 30 seconds after administration. Rabbits to which composition was administered as mist did not display overt signs of ocular irritation.

### Conclusion:

Administration of a composition including an ocular irritant (1% saponin) as a drops caused a decrease in retinal blood flow in two rabbits and no change in a third rabbit, but was accompanied by discomfort.

Administration of a composition including an ocular irritant (1% saponin) as a mist caused a substantial increase in retinal blood flow in two rabbits and a reduced extent of discomfort.

### Example 2

### Effect of Composition Including Benzalkonium Chloride as an Ocular Irritant on Retrobulbar Blood Flow

The effect of compositions including benzalkonium chloride administered in accordance with the teachings herein is performed substantially as described in Example 1. Specifically, four isotonic PBS compositions having pH of 7.4 are prepared having 0.01%, 0.05, 0.1% and 0.2% benzalkonium chloride, respectively, as an ocular irritant.

The compositions are administered to animals and the effect on retrobulbar blood flow is determined, substantially as described above.

It is observed that administration of a composition including a sufficient amount of an ocular irritant (benzalkonium chloride) as a mist causes a substantial increase in retinal blood flow in the animals with little or no substantial discomfort and irritation.

Administration of a composition including an ocular irritant (benzalkonium chloride) as drops cause discomfort and irritation to the animals.

### Example 3

### Exemplary Compositions

A number of exemplary pharmaceutical compositions including an optical irritant in an ophthalmically-acceptable carrier suitable for administration as a mist, include:

Composition 1: an ophthalmically-acceptable carrier comprising standard phosphate buffered saline (PBS) having a pH of 7.4 to which is added 1% saponin as an ocular irritant.

Composition 2: an ophthalmically-acceptable carrier comprising standard phosphate buffered saline (PBS) having a pH of 7.4 to which is added 0.5% deoxycholic acid as an ocular irritant.

Composition 3: an ophthalmically-acceptable carrier comprising standard phosphate buffered saline (PBS) having a pH of 7.4 to which is added 0.1% digitonin as an ocular irritant.

Composition 4: an ophthalmically-acceptable carrier comprising standard phosphate buffered saline (PBS) having a pH of 7.4 to which is added 1% fusidic acid as an ocular irritant.

Composition 5: an ophthalmically-acceptable carrier comprising mannitol (2%), sodium chloride, edetate sodium (0.01%), sodium phosphate dibasic, sodium phosphate monobasic and purified water having a pH of 6.6 to which is added 2% fusidate as an ocular irritant.

Composition 6: an ophthalmically-acceptable carrier comprising as a preservative, mannitol, sodium citrate dihydrate, sodium hydroxide (to adjust pH to 5.6) and purified water to which is added 0.0075% benzalkonium chloride and 2% ammonium glycyrrhizide as an ocular irritant.

Composition 7: an ophthalmically-acceptable carrier comprising, monobasic and dibasic sodium phosphate, sodium hydroxide (to adjust pH) and purified water to which is added 0.01% benzalkonium chloride and 3% Brij 35 as an ocular irritant.

Composition 8: an ophthalmically-acceptable carrier comprising mannitol, polysorbate 80, edetate disodium, sodium hydroxide or hydrochloric acid (to adjust pH to 5.0-6.5) and purified water to which is added 0.015% benzalkonium chloride and 2% cetylpyridium chloride as an ocular irritant.

Composition 9: an ophthalmically-acceptable carrier comprising sodium chloride, sodium dihydrogen phosphate monohydrate, disodium hydrogen phosphate anhydrous (to adjust pH to 6.7) and purified water to which is added 0.02% benzalkonium chloride (0.02%) and 0.5% saponin as an ocular irritant.

Composition 10: an ophthalmically-acceptable carrier comprising citric acid, sodium chloride, sodium citrate and purified water with hydrochloric acid and/or sodium hydroxide to adjust pH) to which is added 0.05% benzalkonium chloride and 0.5% escin as an ocular irritant.

Composition 11: an ophthalmically-acceptable carrier comprising monobasic and dibasic sodium phosphate, sodium hydroxide (to adjust pH) and purified water to which is added 0.1% benzalkonium chloride and 3% Brij 35 as an ocular irritant.

Composition 12: an ophthalmically-acceptable carrier comprising mannitol, polysorbate 80, edetate disodium, sodium hydroxide or hydrochloric acid (to adjust pH to 5.0-6.5) and purified water to which is added 0.1% benzalkonium chloride and 2% cetylpyridium chloride as an ocular irritant.

Composition 13: an ophthalmically-acceptable carrier comprising as a preservative and as an ocular irritant, sodium chloride, sodium dihydrogen phosphate monohydrate, disodium hydrogen phosphate anhydrous (to adjust pH to 6.7) and purified water to which is added 0.2% benzalkonium chloride and 0.5% saponin.

Composition 14: an ophthalmically-acceptable carrier comprising citric acid, sodium chloride, sodium citrate and purified water with hydrochloric acid and/or sodium hydroxide to adjust pH) to which is added 0.4% benzalkonium chloride and 0.5% escin as an ocular irritant.

Composition 15: an ophthalmically-acceptable carrier comprising isotonic PBS to which is added 0.01% benzalkonium chloride as an ocular irritant.

Composition 16: an ophthalmically-acceptable carrier comprising citric acid, sodium chloride, sodium citrate and purified water with hydrochloric acid and/or sodium hydroxide to adjust pH) to which is added 0.5% sodium caprate as an ocular irritant.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, many alternatives, modifications and variations will be apparent to one skilled in the art upon perusal of the description and the Figures. Accordingly, the present invention embraces all such alternatives, modifications and variations that fall within the spirit and broad scope of the appended claims.

Section headings are used herein to ease understanding of the specification and should not be construed as necessarily limiting.

1-21. (canceled)

22. A method of treating a condition associated with insufficient retrobulbar blood flow, the method comprising administering to an eye of a subject suffering from a condition associated with insufficient retrobulbar blood flow an effective amount of a pharmaceutical composition in the form of a mist consisting essentially of an ocular irritant and an ophthalmically-acceptable carrier, whereby the ocular irritant stimulates retrobulbar blood flow, thereby treating the condition.

23. The method of claim 22, wherein administering of the pharmaceutical composition in the form of a mist occurs with reduced, minimal or no irritation to the eye.

24. The method of claim 22, wherein administering comprises contacting an anterior surface of the eye with the mist.

25. The method of claim 24, wherein administering the mist leads to depositing an amount of the ocular irritant on a posterior surface of the eye effective in stimulating the retrobulbar blood flow.

26. The method of claim 22, wherein the pharmaceutical composition is substantially devoid of an active pharmaceutical ingredient other than the ocular irritant.

27. The method of claim 22, wherein the condition is selected from diabetic retinopathy, open angle glaucoma, ocular hypertension, macular degeneration, ocular ischemic syndrome, giant cell arteritis, eye occlusions, central retinal artery occlusion (CRAO), central retinal vein occlusion (CRVA), ischemic optic neuropathy, optic neuritis, neuromyelitis optica and neuroretinitis.

28. The method of claim 22, wherein the ocular irritant is selected from saponin, benzalkonium chloride or both.

29. The method of claim 22, wherein the mist comprises particles having a mean particle diameter of less than about 20 microns.

30. The method of claim 22, wherein the mist comprises particles having a mean particle diameter of less than about 5 microns.

31. A method of treatment, comprising:
a) providing a pharmaceutical composition consisting essentially of an ocular irritant and an ophthalmically-acceptable carrier;
b) generating a mist of said pharmaceutical composition; and
c) contacting said mist with a posterior surface of an eye of a subject in need thereof.

32. A pharmaceutical composition consisting essentially of an ocular irritant in an ophthalmically-acceptable carrier, the composition being adapted for administration to an eye as a mist and further adapted for stimulating retrobulbar blood flow.

33. The composition of claim 32, further adapted to cause reduced, minimal or no irritation to the eye.

34. The composition of claim 32, substantially devoid of an active pharmaceutical ingredient other than the ocular irritant.

35. The composition of claim 32, wherein the ocular irritant comprises saponin, benzalkonium chloride or both.

36. The composition of claim 32, wherein the mist comprises particles having a mean particle diameter of less than about 20 microns.

37. The composition of claim 32, wherein the mist comprises particles having a mean particle diameter of less than about 5 microns.

38. The composition of claim 32, further comprising at least one component selected from bioadhesives, buffering agents, chelating agents, humectants, pH-adjusting agents, preservatives, solubilizers, viscosity modifiers and vitamins.

39. A device for ophthalmic administration of a pharmaceutical composition as a mist, the device comprising:
a) a composition-reservoir configured to be functionally associated with a nebulizer; and
b) a pharmaceutical composition consisting essentially of an ocular irritant and an ophthalmically-acceptable carrier contained within said reservoir, said composition is adapted for stimulating retrobulbar blood flow in an eye to which administered as a mist.

40. The device of claim 39, further comprising:
c) a nebulizer configured to nebulize said composition contained in said composition-reservoir and to generate an ophthalmically administrable mist.

41. The device of claim 39, configured to produce a mist comprising particles having a mean particle diameter of less than about 20 microns.

42. The device of claim 39, configured to produce a mist comprising particles having a mean particle diameter of less than about 5 microns.

43. The device of claim 39, wherein said composition is further adapted to cause reduced, minimal or no irritation to the eye.

44. The device of claim 39, wherein said composition is substantially devoid of an active pharmaceutical ingredient other than the ocular irritant.

45. The device of claim 39, wherein said ocular irritant comprises saponin, benzalkonium chloride or both.
